(19) United States
(12) Patent Application Publication
(10) Pub. No.: US 2007/0251027 A1
(43) Pub. Date: Nov. 1, 2007

(54) Dyeing composition comprising at least one soluble fluorescent compound, at least one electrophilic monomer and at least one liquid organic solvent

(76) Inventors: Maryse Chaisy, Goussainville (FR); Luc Gourlaouen, Asnieres (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/783,923

(22) Filed: Apr. 13, 2007

Related U.S. Application Data
(60) Provisional application No. 60/796,931, filed on May 3, 2006.

(30) Foreign Application Priority Data
Apr. 13, 2006 (FR) 0603321

(51) Int. Cl. A61K 8/00

(52) U.S. Cl. 8/405

(57) ABSTRACT

The present disclosure relates to a composition for dyeing keratinous fibers comprising at least one electrophilic monomer, at least one soluble fluorescent compound and at least one liquid organic solvent. The composition in accordance with the present disclosure makes it possible to improve the visibility of the coloring on dark keratinous fibers while exhibiting an improved resistance to external agents.

This application claims benefit of U.S. Provisional Application No. 60/796,931, filed May 3, 2006, the contents of which are incorporated herein by reference. This application also claims benefit of priority under 35 U.S.C. § 119 to French Patent Application No. FR 06/03321, filed Apr. 13, 2006, the contents of which are also incorporated herein by reference.

The present relates to a composition for dyeing keratinous fibers, such as the hair, comprising at least one soluble fluorescent compound, at least one electrophilic monomer and at least one liquid organic solvent.

It is known to dye keratinous fibers, such as human keratinous fibers, by direct dyeing. The process conventionally used in direct dyeing comprises applying, to keratinous fibers, direct dyes which are colored and coloring molecules having an affinity for the fibers, allowing them to diffuse and then rinsing the fibers.

The direct dyes which are conventionally used are, for example, dyes of the nitrobenzene type, anthraquinone dyes, nitropyridines, or dyes of the azo, xanthene, acridine, azine or triarylmethane type.

The colorings which result from the use of direct dyes are temporary or semipermanent colorings since the interactions which bind the direct dyes to the keratinous fiber and their desorption from the surface and/or from the core of the fiber may result in weak dyeing power and poor resistance to washing operations or to perspiration.

Furthermore, the coloring of keratinous fibers starting from conventional direct dyes generally does not make it possible to significantly lighten keratinous fibers.

The lightening of the color of keratinous fibers constitutes an important demand in the market.

Conventionally, in order to obtain a lighter coloring, a chemical bleaching process is employed. This process comprises bleaching the melanins present in the fibers by an oxidizing system composed of hydrogen peroxide, in combination or not with persalts, in an alkaline medium.

This bleaching system exhibits the disadvantage of damaging the fibers and of detrimentally affecting their cosmetic properties. This is because the hair has a tendency to become rough, more difficult to disentangle and more brittle. Thus, the lightening of the hair is often accompanied by a significant decline in the mechanical qualities of the hair.

Finally, the lightening or the bleaching of keratinous fibers starting from an oxidizing agent can be incompatible with the treatments for modifying the shape of the fibers, for instance in hair straightening treatments.

Another known lightening technique comprises applying, to dark hair, direct fluorescent dyes. This technique described, for example, in French Patent No. 2 830 189, makes it possible to retain the quality of the keratinous fiber during the treatment but the fluorescent dyes employed do not exhibit good resistance to shampooing operations.

Furthermore, compositions for the treatment of hair starting from compositions comprising electrophilic monomers are known, for example, from French Patent Application No. 2 833 489. Such a composition makes it possible to obtain completely sheathed and nongreasy hair.

Disclosed herein, therefore, are novel compositions for dyeing keratinous fibers, such as the hair, which make it possible to obtain visible colorings on dark hair without it being necessary to lighten or to bleach the fibers and which exhibit good resistance to external agents.

The present disclosure also relates to a dyeing composition comprising at least one electrophilic monomer, at least one soluble fluorescent compound and at least one liquid organic solvent.

The composition in accordance with the present disclosure makes it possible to improve the visibility of the coloring, such as on dark hair. In the case of dark keratinous fibers, a highly visible coloring is obtained without it being necessary to lighten or to bleach the keratinous fibers and consequently without physically damaging the keratinous fibers. Thus, this composition makes it possible to obtain optical lightening effects on dark hair, for instance hair which exhibits a tone height of less than or equal to 6.

The use of the presently disclosed fluorescent compound in a dyeing composition also makes it possible to obtain colorings with special effects under the effect of light rich in ultra violet radiation, such as the lighting effects used in nightclubs, for example.

Furthermore, this coloring exhibits good resistance to the various assaults to which hair may be subjected, such as shampooing operations, rubbing, light, bad weather, sweat and permanent deformations. In at least one embodiment, the coloring exhibits good resistance to shampooing operations.

The present disclosure also relates to a process for dyeing keratinous fibers comprising applying to the keratinous fibers the composition of the disclosure and also the use of the composition in the dyeing of keratinous fibers, such as the hair.

The present disclosure additionally relates to a kit comprising a composition comprising at least one fluorescent compound as defined herein and another composition comprising at least one electrophilic monomer, it being possible for the liquid organic solvent to be in either of the compositions.

As used herein, the term “soluble fluorescent compound” is understood to mean a compound which exhibits a solubility in the medium of greater than 0.0001% at 20° C., for instance greater than 0.01%.

Furthermore, as used herein the term “fluorescent compound” is understood to mean fluorescent dyes and optical brighteners.

Fluorescent dyes are compounds which absorb visible radiation, ranging from 400 to 800 nm, and which are capable of re-emitting light in the visible region at a higher wavelength. By definition, these dyes are colored entities since they absorb visible light.

According to at least one embodiment of the present disclosure, the fluorescent dyes re-emit orange-coloured fluorescent light. They exhibit, for example,a maximum re-emission wavelength ranging from 500 to 700 nm.

Non-limiting mention may be made of fluorescent dyes described in the following works: Ullmann's Encyclopedia of Industrial Chemistry, Release 2004, 7th edition, “Fluorescent Dyes” chapter.

The optical brighteners used in the present disclosure are also known under the name of “brighteners”, or “fluorescent brighteners”, or “fluorescent brightening agents” or “FWA”, or “fluorescent whitening agents”, or “whiteners”, or “fluorescent whiteners.” Optical brighteners are colorless transparent compounds as they do not absorb in visible light but only in ultraviolet light (wavelengths ranging from 200 to 400 nanometers) and convert the energy absorbed into fluorescent light of higher wavelength emitted in the visible part of the spectrum, generally in the blue and/or green, i.e., in wavelengths ranging from 400 to 550 nanometers.

Optical brighteners are known in the art and described, for example, in Ullmann's Encyclopedia of Industrial Chemistry (2002), “Optical Brighteners” and Kirk-Othmer Encyclopedia of Chemical Technology (1995): “Fluorescent Whitening Agents”.

The fluorescent dyes which can be used in the context of the present disclosure are compounds known in the art. They are, for example, described in French Patent No. 2 830 189. Further non-limiting examples of fluorescent dyes include:
Photosensitizing Dye NK-557, sold by Ubichem, which exhibits the following structure:

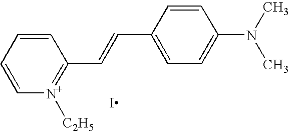

### 2-[2-(4-(dimethylamino)phenyl)ethenyl]-1-ethylpyridinium iodide;

Brilliant Yellow B6GL, sold by Sandoz and with the following structure: 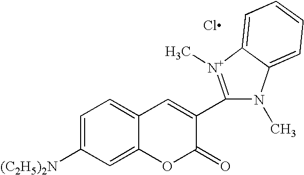
Basic Yellow 2 or Auramine O sold by Prolabo, Aldrich or Carlo Erba and with the following structure:

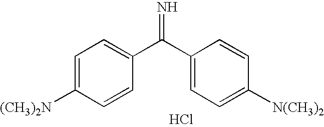

### 4,4′-(imidocarbonyl)bis(N,N-dimethylaniline)monohydrochloride

Additional non-limiting examples of optical brighteners and fluorescent dyes, as disclosed herein, include:
Naphthalimides, for example the following compound:

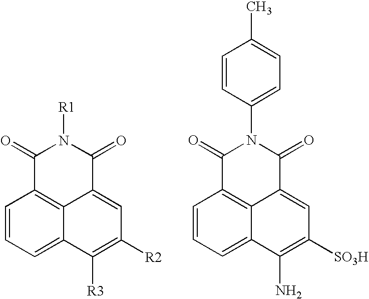

Brilliant Sulphoflavin FF, C.I. 56205

wherein R1, R2 and R3 are chosen from, independently of one another, hydrogen; halogen atoms; C₆-C₃₀ aryl groups; hydroxyl groups; cyano groups; nitro groups; sulpho groups; amino groups; acylamino groups; di(C₁-C₆)alkylamino groups; dihydroxy(C₁-C₆)alkylamino groups; (C₁-C₆)alkylhydroxy(C₁-C₆)alkylamino groups; (C₁-C₆)alkoxy groups; (C₁-C₆)alkoxycarbonyl groups; carboxy(C₁-C₆)alkoxy groups; piperidinosulphonyl groups; pyrrolidino groups; (C₁-C₆)alkylhalo(C₁-C₆)alkylamino groups; benzoyl(C₁-C₆)alkyl groups; vinyl groups; formyl groups; C₆-C₃₀ aryl radicals optionally substituted by at least one group chosen from a hydroxyl group, a linear, branched or cyclic C₁-C₆ alkoxy group and a linear, branched or cyclic alkyl group comprising from 1 to 22 carbon atoms, itself optionally being substituted by at least one group chosen from hydroxyl, amino and C₁-C₆ alkoxy groups; and linear, branched or cyclic alkyl radicals comprising from 1 to 22 carbon atoms, for instance from 1 to 6 carbon atoms, optionally substituted by at least one group chosen from hydroxyl, amino, linear, branched and cyclic C₁-C₆ alkoxy, optionally substituted aryl, carboxyl or sulpho groups and a halogen atom, it being possible for this alkyl radical to be interrupted by a heteroatom, such as a nitrogen, sulfur and oxygen atom; and further wherein the R1, R2 and R3 substituents can form, with the carbon atoms to which they are attached, a C₆-C₃₀ aromatic or nonaromatic ring or a heterocyclic ring comprising, in total, from 5 to 30 ring members and from 1 to 5 heteroatoms; these rings being fused or nonfused and inserting or not inserting a carbonyl group and being unsubstituted or substituted by at least one group chosen from C₁-C₄ alkyl, (C₁-C₄)alkoxy(C₁-C₄)alkyl, amino, di(C₁-C₄)alkylamino, halogen, phenyl, carboxyl and tri(C₁-C₄)alkyl-ammonio(C₁-C₄)alkyl groups.
Coumarin derivatives, such as the compounds corresponding to the following formulae: 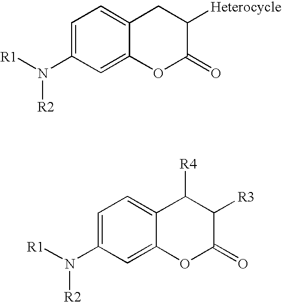
wherein the heterocycle is chosen from furan, thiophene, 2H-pyrrole, 2-pyrroline, pyrrolidine, 1,3-dioxolane, oxazole, thiazole, imidazole, 2-imidazoline, imidazolidine, pyrazole, 2-pyrazoline, pyrazolidine, isoxazole, isothiazole, 1,2,3-oxadiazole, 1,2,3-triazole, 1,3,4-thiadiazole, 2H-pyran, 4H-pyran, pyridine, piperidine, 1,4-dioxane, morpholine, 1,4-dithiane, thiomorpholine, pyridazine, pyrimidine, pyrazine, piperazine, 1,3,5-triazine, 1,3,5-trithiane, indolizine, indole, isoindole, 3H-indole, indoline, benzo[b]furan, benzo[b]thiophene, 1H-indazole, benzimidazole, benzothiazole, purine, 4H-quinolizine, quinoline, isoquinoline, cinnoline, phthalazine, quinazoline, quinoxaline, 1,8-naphthyridine, pteridine, quinuclidine, carbazole, acridine, phenazine, phenothiazine, phenoxazine, indene, naphthalene, azulene, fluorene, anthracene, norbornane, and adamantane;
and wherein R1, R2, R3 and R4 are chosen from, independently of one another, hydrogen; halogen atoms; C₆-C₃₀ aryl groups; hydroxyl groups; cyano groups; nitro groups; sulpho groups; amino groups; acylamino groups; di(C₁-C₆)alkylamino groups; dihydroxy-(C₁-C₆)alkylamino groups; (C₁-C₆)alkylhydroxy(C₁-C₆)alkylamino groups; (C₁-C₆)alkoxy groups; (C₁-C₆)alkoxycarbonyl groups; carboxy(C₁-C₆)alkoxy groups; piperidinosulphonyl groups; pyrrolidino groups; (C₁-C₆)alkylhalo(C₁-C₆)alkylamino groups; benzoyl(C₁-C₆)alkyl groups; vinyl groups; formyl groups; and C₆-C₃₀ aryl radicals optionally substituted by at least one group chosen from a hydroxyl group, a linear, branched or cyclic C₁-C₆ alkoxy group and a linear, branched or cyclic alkyl group comprising from 1 to 22 carbon atoms, itself optionally being substituted by at least one group chosen from hydroxyl, amino and C₁-C₆ alkoxy groups; a linear, branched and cyclic alkyl radical comprising from 1 to 22 carbon atoms, for instance from 1 to 6 carbon atoms, optionally substituted by at least one group chosen from hydroxyl, amino, linear, branched and cyclic C₁-C₆ alkoxy, optionally substituted aryl, carboxyl or sulpho groups or a halogen atom, it being possible for this alkyl radical to be interrupted by a heteroatom, such as a nitrogen, sulphur and oxygen atom;
further wherein the R1, R2, R3 and R4 substituents can form, with the carbon atoms to which they are attached, a C₆-C₃₀ aromatic or nonaromatic ring or a heterocyclic ring comprising, in total, from 5 to 30 ring members and from 1 to 5 heteroatoms; these rings being fused or nonfused and inserting or not inserting a carbonyl group and being unsubstituted or substituted by at least one group chosen from C₁-C₄ alkyl, (C₁-C₄)alkoxy(C₁-C₄)alkyl, amino, di(C₁-C₄)alkylamino, halogen, phenyl, carboxyl and tri(C₁-C₄)alkylammonio(C₁-C₄)alkyl groups;
and wherein two of the R3 and R4 substituents can form, with the carbon atoms to which they are attached, a C₆-C₃₀ aromatic ring or a heterocyclic ring comprising, in total, from 5 to 30 ring members and from 1 to 5 heteroatoms; this ring being fused or nonfused, this ring and the possible fused ring being unsubstituted or substituted by at least one group chosen from C₁-C₄ alkyl, (C₁-C₄)alkoxy(C₁-C₄)alkyl, amino, di(C₁-C₄)alkylamino, halogen, phenyl, carboxyl and tri(C₁-C₄)alkylammonio(C₁-C₄)alkyl groups.
Mention may be made, by way of non-limiting example, of: 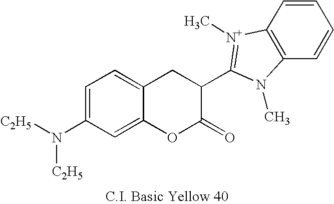
Xanthene derivatives, such as: 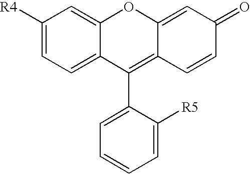
wherein R4 and R5 are chosen from, independently of one another, hydrogen; halogen atoms; C₆-C₃₀ aryl groups; hydroxyl groups; cyano groups; nitro groups; sulpho groups; amino groups; acylamino groups; di(C₁-C₆)alkylamino groups; dihydroxy(C₁-C₆)alkylamino groups; (C₁-C₆)alkylhydroxy(C₁-C₆)alkylamino groups; (C₁-C₆)alkoxy group; (C₁-C₆)alkoxycarbonyl groups; carboxy(C₁-C₆)alkoxy groups; piperidinosulphonyl groups; pyrrolidino groups; (C₁-C₆)alkylhalo(C₁-C₆)alkylamino groups; benzoyl(C₁-C₆)alkyl groups; vinyl groups; formyl groups; C₆-C₃₀ aryl radicals optionally substituted by at least one group chosen from a hydroxyl group, a linear, branched or cyclic C₁-C₆ alkoxy group and a linear, branched or cyclic alkyl group comprising from 1 to 22 carbon atoms, itself optionally being substituted by at least one group chosen from hydroxyl, amino and C₁-C₆ alkoxy groups; and linear, branched or cyclic alkyl radicals comprising from 1 to 22 carbon atoms, for instance from 1 to 6 carbon atoms, optionally substituted by at least one group chosen from hydroxyl, amino, linear, branched and cyclic C₁-C₆ alkoxy, optionally substituted aryl, carboxyl or sulpho groups and a halogen atom, it being possible for this alkyl radical to be interrupted by a heteroatom, such as nitrogen, sulphur and oxygen.
Rhodamines, such as: 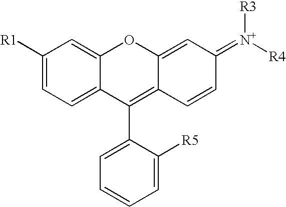
wherein R1, R2, R3 and R4 are as previously defined. Mention may be made, by way of non-limiting example, of 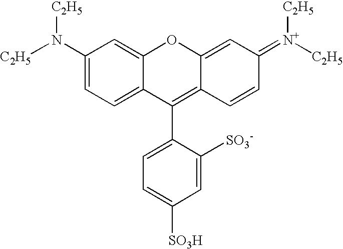
Sulphorhodamine B C.I. 45100 Acid Red 52
Thioxanthene derivatives, such as: 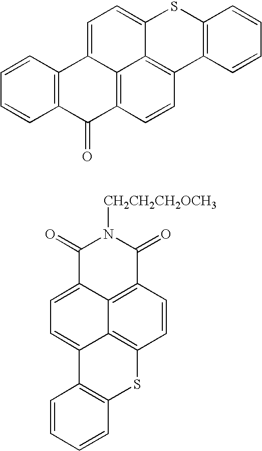
Samaron, Brilliant Yellow H6GL, C.I. 56235 Disperse Yellow 105
Naphtholactam derivatives, such as: 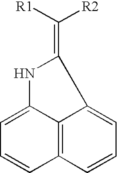
with R1 and R2 as previously defined. Mention may be made, for example, of the following compound:

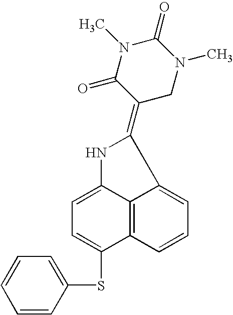

Disperse Dye 28
Azalactone derivatives: 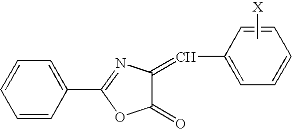
wherein X has the same definition as R1 as previously defined. Mention may be made, for example, of the following compound: 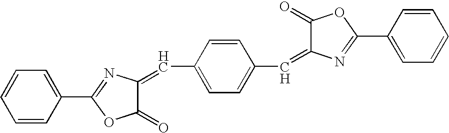
Methine derivatives, such as: 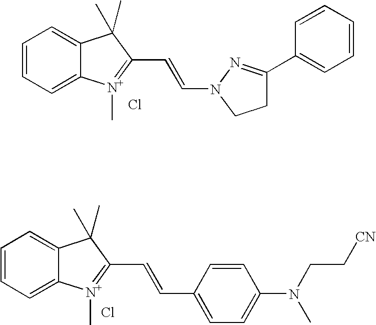
Oxazine and thiazine derivatives, such as: 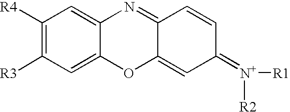
wherein R1, R2, R3 and R4 are previously defined. Mention may be made, by way of example, of: 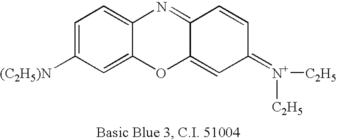
1,4-Distyrylbenzene derivatives of formula: 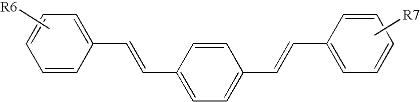
wherein R6 and R7 are chosen from, independently of one another, hydrogen; halogen atoms; C₆-C₃₀ aryl groups; hydroxyl groups; cyano groups; nitro groups; sulpho groups; amino groups; acylamino groups; di(C₁-C₆)alkylamino groups; dihydroxy(C₁-C₆)alkylamino groups; (C₁-C₆)alkylhydroxy(C₁-C₆)alkylamino groups; (C₁-C₆)alkoxy groups; (C₁-C₆)alkoxycarbonyl groups; carboxy(C₁-C₆)alkoxy groups; piperidinosulphonyl groups; pyrrolidino groups; (C₁-C₆)alkylhalo(C₁-C₆)alkylamino groups; benzoyl(C₁-C₆)alkyl groups; vinyl groups; formyl groups; C₆-C₃₀ aryl radicals optionally substituted by at least one group chosen from a hydroxyl group, a linear, branched or cyclic C₁-C₆ alkoxy group and a linear, branched or cyclic alkyl group comprising from 1 to 22 carbon atoms, itself optionally being substituted by at least one group chosen from hydroxyl, amino and C₁-C₆ alkoxy groups; and linear, branched or cyclic alkyl radicals comprising from 1 to 22 carbon atoms, for instance from 1 to 6 carbon atoms, optionally substituted by at least one group chosen from hydroxyl, amino, linear, branched and cyclic C₁-C₆ alkoxy, optionally substituted aryl, carboxyl or sulpho groups and a halogen atom, it being possible for this alkyl radical to be interrupted by a heteroatom, such as nitrogen, sulfur and oxygen.
4,4′-Distyrylbiphenyl derivatives of formula: 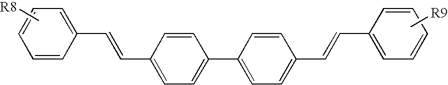
wherein R8 and R9 are chosen from, independently of one another, hydrogen; halogen atoms; C₆-C₃₀ aryl groups; hydroxyl groups; cyano groups; nitro groups; sulpho groups; amino groups; acylamino groups; di(C₁-C₆)alkylamino groups; dihydroxy(C₁-C₆)alkylamino groups; (C₁-C₆)alkylhydroxy(C₁-C₆)alkylamino groups; (C₁-C₆)alkoxy groups; (C₁-C₆)alkoxycarbonyl groups; carboxy(C₁-C₆)alkoxy groups; piperid inosulphonyl groups; pyrrolidino groups; (C₁-C₆)alkylhalo(C₁-C₆)alkylamino groups; benzoyl(C₁-C₆)alkyl groups; vinyl groups; formyl groups; C₆-C₃₀ aryl radicals optionally substituted by at least one group chosen from a hydroxyl group, a linear, branched or cyclic C₁-C₆ alkoxy group and a linear, branched or cyclic alkyl group comprising from 1 to 22 carbon atoms, itself optionally being substituted by at least one group chosen from hydroxyl, amino and C₁-C₆ alkoxy groups; and linear, branched or cyclic alkyl radicals comprising from 1 to 22 carbon atoms, for example from 1 to 6 carbon atoms, optionally substituted by at least one group chosen from hydroxyl, amino, linear, branched and cyclic C₁-C₆ alkoxy, optionally substituted aryl, carboxyl or sulpho groups and a halogen atom, it being possible for this alkyl radical to be interrupted by a heteroatom, such as nitrogen, sulphur and oxygen.
Triazinylaminostilbene derivatives of formula: 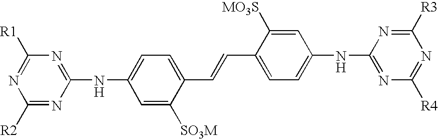
wherein R1, R2, R3 and R4 are chosen from, independently of one another, hydrogen; halogen atoms; C₆-C₃₀ aryl groups; hydroxyl groups; cyano groups; nitro groups; sulpho groups; amino groups; acylamino groups; di(C₁-C₆)alkylamino groups; dihydroxy(C₁-C₆)alkylamino groups; (C₁-C₆)alkylhydroxy(C₁-C₆)alkylamino groups; (C₁-C₆)alkoxy groups; (C₁-C₆)alkoxycarbonyl groups; carboxy(C₁-C₆)alkoxy groups; piperidinosulphonyl groups; pyrrolidino groups; (C₁-C₆)alkylhalo(C₁-C₆)alkylamino groups; benzoyl(C₁-C₆)alkyl groups; vinyl groups; formyl groups; C₆-C₃₀ aryl radicals optionally substituted by at least one group chosen from a hydroxyl group, a linear, branched or cyclic C₁-C₆ alkoxy group and a linear, branched or cyclic alkyl group comprising from 1 to 22 carbon atoms, itself optionally being substituted by at least one group chosen from hydroxyl, amino and C₁-C₆ alkoxy groups; and a linear, branched or cyclic alkyl radical comprising from 1 to 22 carbon atoms, for instance from 1 to 6 carbon atoms, optionally substituted by at least one group chosen from hydroxyl, amino, linear, branched and cyclic C₁-C₆ alkoxy, optionally substituted aryl, carboxyl or sulpho groups or a halogen atom, it being possible for this alkyl radical to be interrupted by a heteroatom, such as nitrogen, sulphur and oxygen;
and M is a monovalent or divalent cation resulting from the family of alkali metals or alkaline earth metals, such assodium, potassium and calcium ions.
Stilbazolium derivatives of formula: 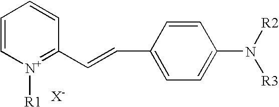
wherein R1, R2 and R3 are chosen from, independently of one another, hydrogen; halogen atoms; C₆-C₃₀ aryl groups; hydroxyl groups; cyano groups; nitro groups; sulpho groups; amino groups; acylamino groups; di(C₁-C₆)alkylamino groups; dihydroxy(C₁-C₆)alkylamino groups; (C₁-C₆)alkylhydroxy(C₁-C₆)alkylamino groups; (C₁-C₆)alkoxy groups; (C₁-C₆)alkoxycarbonyl groups; carboxy(C₁-C₆)alkoxy groups; piperidinosulphonyl groups; pyrrolidino groups; (C₁-C₆)alkylhalo(C₁-C₆)alkylamino groups; benzoyl(C₁-C₆)alkyl groups; vinyl groups; formyl groups; C₆-C₃₀ aryl radicals optionally substituted by at least one group chosen from a hydroxyl group, a linear, branched or cyclic C₁-C₆ alkoxy group and linear, branched or cyclic alkyl groups comprising from 1 to 22 carbon atoms, itself optionally being substituted by at least one group chosen from hydroxyl, amino and C₁-C₆ alkoxy groups; and a linear, branched or cyclic alkyl radical comprising from 1 to 22 carbon atoms, for example from 1 to 6 carbon atoms, optionally substituted by at least one group chosen from hydroxyl, amino, linear, branched and cyclic C₁-C₆ alkoxy, optionally substituted aryl, carboxyl or sulpho groups or a halogen atom, it being possible for this alkyl radical to be interrupted by a heteroatom, such as nitrogen, sulphur and oxygen;
two of the R2 and R3 substituents can form, with the carbon atoms to which they are attached, C₆-C₃₀ aromatic rings or heterocyclic rings comprising, in total, from 5 to 30 ring members and from 1 to 5 heteroatoms; this ring being fused or nonfused, this ring and the possible fused ring being unsubstituted or substituted by at least one group chosen from C₁-C₄ alkyl, (C₁-C₄)alkoxy(C₁-C₄)alkyl, amino, di(C₁-C₄)alkylamino, halogen, phenyl, carboxyl and tri(C₁-C₄)alkylammonio(C₁-C₄)alkyl groups;
X⁻ is an organic or inorganic anion. Non-limiting examples of organic or inorganic anions include: chloride, bromide, iodide, methosulphate, ethosulphate, mesylate, tosylate or acetate ions or simple organic acid salts, such as lactates, oleates, benzoates, perchlorates or triflates.
Stilbazolium dimers of formulae: 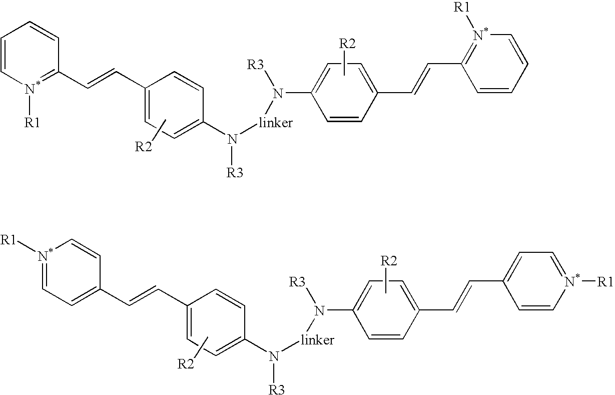
wherein R1, R2 and R3 are chosen from, independently of one another, hydrogen; halogen atoms; C₆-C₃₀ aryl groups; hydroxyl groups; cyano groups; nitro groups; sulpho groups; amino groups; acylamino groups; di(C₁-C₆)alkylamino groups; dihydroxy(C₁-C₆)alkylamino groups; (C₁-C₆)alkylhydroxy(C₁-C₆)alkylamino groups; (C₁-C₆)alkoxy groups; (C₁-C₆)alkoxycarbonyl groups; carboxy(C₁-C₆)alkoxy groups; piperidinosulphonyl groups; pyrrolidino groups; (C₁-C₆)alkylhalo(C₁-C₆)alkylamino groups; benzoyl(C₁-C₆)alkyl groups; vinyl groups; formyl groups; C₆-C₃₀ aryl radical optionally substituted by at least one group chosen from a hydroxyl group, a linear, branched or cyclic C₁-C₆ alkoxy group and a linear, branched or cyclic alkyl group comprising from 1 to 22 carbon atoms, itself optionally being substituted by at least one group chosen from hydroxyl, amino and C₁-C₆ alkoxy groups; and linear, branched or cyclic alkyl radicals comprising from 1 to 22 carbon atoms, for example from 1 to 6 carbon atoms, optionally substituted by at least one group chosen from hydroxyl, amino, linear, branched and cyclic C₁-C₆ alkoxy, optionally substituted aryl, carboxyl or sulpho groups or a halogen atom, it being possible for this alkyl radical to be interrupted by a heteroatom, such as nitrogen, sulphur and oxygen.
The following stilbazolium trimers and tetramers: 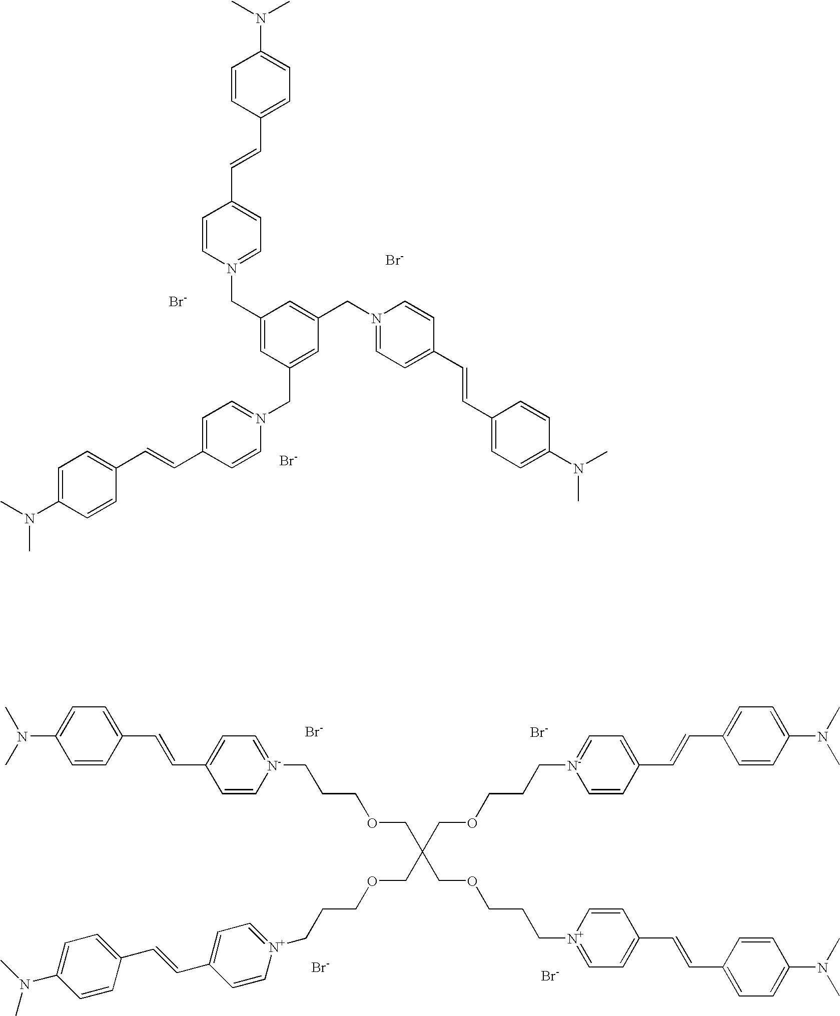 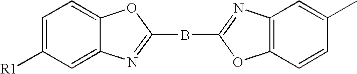
wherein R1 and R2 are chosen from, independently of one another, hydrogen; halogen atoms; C₆-C₃₀ aryl groups; hydroxyl groups; cyano groups; nitro groups; sulpho groups; amino groups; acylamino groups; di(C₁-C₆)alkylamino groups; dihydroxy(C₁-C₆)alkylamino groups; (C₁-C₆)alkylhydroxy(C₁-C₆)alkylamino groups; (C₁-C₆)alkoxy groups; (C₁-C₆)alkoxycarbonyl groups; carboxy(C₁-C₆)alkoxy groups; piperidinosulphonyl groups; pyrrolidino groups; (C₁-C₆)alkylhalo(C₁-C₆)alkylamino groups; benzoyl(C₁-C₆)alkyl groups; vinyl groups; formyl groups; C₆-C₃₀ aryl radical optionally substituted by at least one group chosen from a hydroxyl group, a linear, branched or cyclic C₁-C₆ alkoxy group and a linear, branched or cyclic alkyl group comprising from 1 to 22 carbon atoms, itself optionally being substituted by at least one group chosen from hydroxyl, amino and C₁-C₆ alkoxy groups; and linear, branched or cyclic alkyl radicals comprising from 1 to 22 carbon atoms, for example from 1 to 6 carbon atoms, optionally substituted by at least one group chosen from hydroxyl, amino, linear, branched and cyclic C₁-C₆ alkoxy, optionally substituted aryl, carboxyl or sulpho groups or a halogen atom, it being possible for this alkyl radical to be interrupted by a heteroatom, such as nitrogen, sulphur and oxygen; and
B is chosen from: 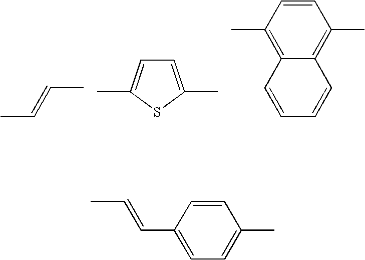
Cationic or noncationic bis(benzimidazoles);
Anionic or nonanionic 1,3-diphenyl-2-pyrazolines, for example: 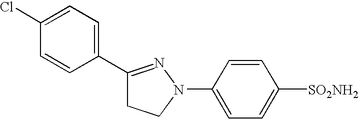
Diketopyrrolopyrroles of formula: 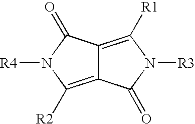
wherein R1, R2, R3 and R4 are chosen from, independently of one another, hydrogen; halogen atoms; C₆-C₃₀ aryl groups; hydroxyl groups; cyano groups; nitro groups; sulpho groups; amino groups; acylamino groups; di(C₁-C₆)alkylamino groups; dihydroxy(C₁-C₆)-alkylamino groups; (C₁-C₆)alkylhydroxy(C₁-C₆)alkylamino groups; (C₁-C₆)alkoxy groups; (C₁-C₆)alkoxycarbonyl groups; carboxy(C₁-C₆)alkoxy groups; piperidinosulphonyl groups; pyrrolidino groups; (C₁-C₆)alkylhalo(C₁-C₆)alkylamino groups; benzoyl(C₁-C₆)alkyl groups; vinyl groups; formyl groups; C₆-C₃₀ aryl radicals optionally substituted by at least one group chosen from a hydroxyl group, a linear, branched or cyclic C₁-C₆ alkoxy group and a linear, branched or cyclic alkyl group comprising from 1 to 22 carbon atoms, itself optionally being substituted by at least one group chosen from hydroxyl, amino and C₁-C₆ alkoxy groups; and linear, branched or cyclic alkyl radicals comprising from 1 to 22 carbon atoms, for example from 1 to 6 carbon atoms, optionally substituted byat least one group chosen from hydroxyl, amino, linear, branched and cyclic C₁-C₆ alkoxy, optionally substituted aryl, carboxyl or sulpho groups or a halogen atom, it being possible for this alkyl radical to be interrupted by a heteroatom, such as nitrogen, sulphur and oxygen.

Non-limiting mention may be made, for example, of the following compound: 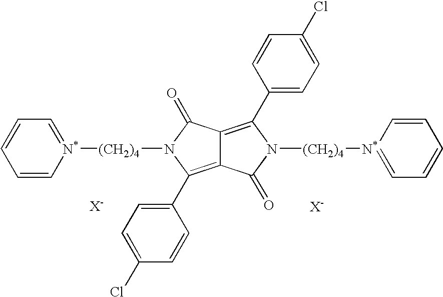
wherein X⁻ is an anion as previously defined.
Dicyanopyrazine derivatives of formulae: 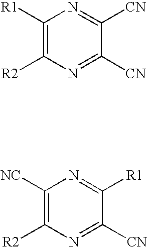
wherein R1 and R2 are chosen from, independently of one another, hydrogen; halogen atoms; C₆-C₃₀ aryl groups; hydroxyl groups; cyano groups; nitro groups; sulpho groups; amino groups; acylamino groups;di(C₁-C₆)alkylamino groups;dihydroxy(C₁-C₆)alkylamino groups; (C₁-C₆)alkylhydroxy(C₁-C₆)alkylamino groups; (C₁-C₆)alkoxy groups; (C₁-C₆)alkoxycarbonyl groups; carboxy(C₁-C₆)alkoxy groups; piperidinosulphonyl groups; pyrrolidino groups; (C₁-C₆)alkylhalo(C₁-C₆)alkylamino groups; benzoyl(C₁-C₆)alkyl groups; vinyl groups; formyl groups; C₆-C₃₀ aryl radicals optionally substituted by at least one group chosen from a hydroxyl group, a linear, branched or cyclic C₁-C₆ alkoxy group and linear, branched or cyclic alkyl groups comprising from 1 to 22 carbon atoms, itself optionally being substituted by at least one group chosen from hydroxyl, amino and C₁-C₆ alkoxy groups; and a linear, branched or cyclic alkyl radical comprising from 1 to 22 carbon atoms, for example from 1 to 6 carbon atoms, optionally substituted by at least one group chosen from hydroxyl, amino, linear, branched and cyclic C₁-C₆ alkoxy, optionally substituted aryl, carboxyl or sulpho groups or a halogen atom, it being possible for this alkyl radical to be interrupted by a heteroatom, such as nitrogen, sulphur and oxygen;
two of the R1 and R2 substituents can form, with the carbon atoms to which they are attached, C₆-C₃₀ aromatic or nonaromatic rings or heterocyclic rings comprising, in total, from 5 to 30 ring members and from 1 to 5 heteroatoms; these rings being fused or nonfused and inserting or not inserting a carbonyl group and being unsubstituted or substituted by at least one group chosen from C₁-C₄ alkyl, (C₁-C₄)alkoxy(C₁-C₄)alkyl, amino, di(C₁-C₄)alkylamino, halogen, phenyl, carboxyl and tri(C₁-C₄)alkyl-ammonio(C₁-C₄)alkyl groups;
two of the R2 and R3 substituents can form, with the carbon atoms to which they are attached, C₆-C₃₀ aromatic rings or heterocyclic rings comprising, in total, from 5 to 30 ring members and from 1 to 5 heteroatoms; this ring being fused or nonfused, this ring and the possible fused ring being unsubstituted or substituted by at least one group chosen from C₁-C₄ alkyl, (C₁-C₄)alkoxy(C₁-C₄)alkyl, amino, di(C₁-C₄)alkylamino, halogen, phenyl, carboxyl and tri(C₁-C₄)alkylammonio(C₁-C₄)alkyl groups.
Non-limiting mention may be made of the compounds in the following publication: “Selective topochemical photoreaction of crystallized 2,3-(phenylethenyl)-4,5-dicyanopyrazine” by Kim, Jae Hong; Matsuoka Masaru, Chem. Lett. (1999), (2), 143-144.

Further, non-limiting mention may be made of:

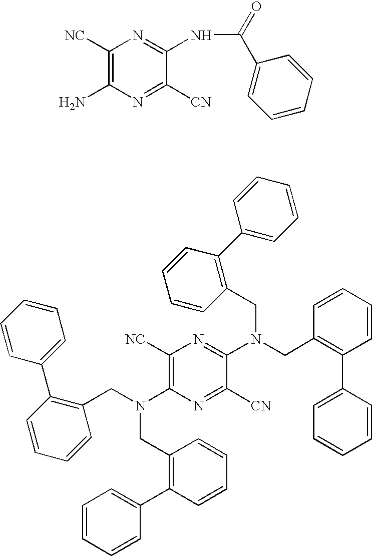

The amount of soluble fluorescent compounds used in the present disclosure can vary within highly varied ranges. By way of example, the amount of fluorescent compounds can range from 0.01 to 40%, for example, from 0.05 to 20% and, further for example, from 0.1 to 10% by weight of the total weight of the composition.

The at least one electrophilic monomer present in the composition of the present disclosure can, for example, be chosen from:
the derivatives benzylidenemalononitrile (A), 2-(4-chlorobenzylidene)malononitrile (A1), ethyl 2-cyano-3-phenylacrylate (B) or ethyl 2-cyano-3-(4-chlorophenyl)acrylate (B1), described in Sayyah, J. Polymer Research, 2000, p 97;

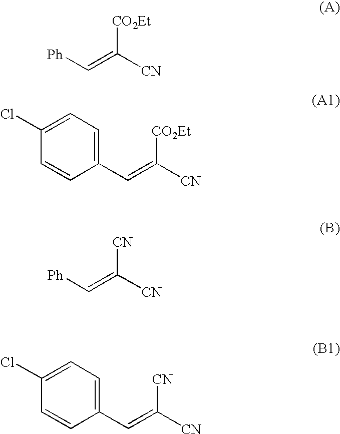

Methylidenemalonate derivatives, such as:
Diethyl 2-methylenemalonate (C), by Hopff, Makromoleculare Chemie, 1961, p 95, De Keyser, J. Pharm. Sci., 1991, p 67, and Klemarczyk, Polymer, 1998, p 173; 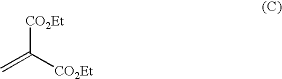
Ethyl 2-ethoxycarbonylmethyloxycarbonylacrylate (D), by Breton, Biomaterials, 1998, p 271 and Couvreur, Pharmaceutical Research, 1994, p 1270; 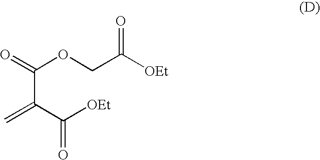
Itaconate and itaconimide derivatives, such as:
Dimethyl itaconate (E), by Bachrach, European Polymer Journal, 1976, p 563; 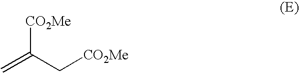
N-Butylitaconimide (F), N-(4-tolyl)itaconimide (G), N-(2-ethylphenyl)itaconimide (H) or N-(2,6-diethylphenyl)itaconimide (I), by Wanatabe, J. Polymer Science: Part A: Polymer Chemistry, 1994, p 2073; 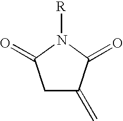
R═Bu (F), 4-tolyl (G), 2-ethylphenyl (H), 2,6-diethylphenyl (I)

The derivatives methyl α-(methylsulphonyl)acrylate (K), ethyl α-(methylsulphonyl)acrylate (L), methyl α-(tert-butylsulphonyl)acrylate (M), tert-butyl α-(methylsulphonyl)acrylate (N) and tert-butyl α-(tert-butylsulphonyl)acrylate (O), by Gipstein, J. Org. Chem., 1980, p 1486, and
the derivatives 1,1-bis(methylsulphonyl)ethylene (P), 1-acetyl-1-(methylsulphonyl)ethylene (Q), methyl α-(methylsulphonyl)vinylsulphonate (R) and α-(methylsulphonyl)acrylonitrile (S), by Shearer, U.S. Pat. No. 2,748,050;

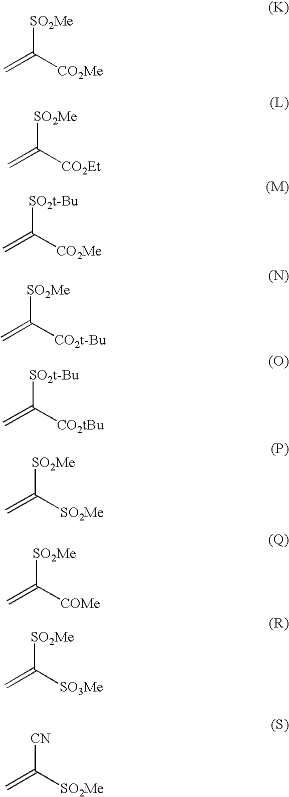

The derivatives methyl vinyl sulphone (T) and phenyl vinyl sulphone (U), by Boor, J. Polymer Science, 1971, p 249;

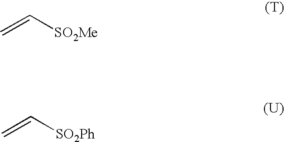

The derivative phenyl vinyl sulphoxide (V), by Kanga, Polymer Preprints (ACS, Division of Polymer Chemistry), 1987, p 322;

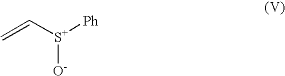

The derivative 3-methyl-N-(phenylsulphonyl)-1-aza-1,3-butadiene (W), by Bonner, Polymer Bulletin, 1992, p 517;

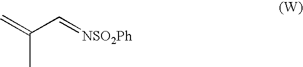

Acrylate and acrylamide derivatives, such as:
N-propyl-N-(3-triisopropoxysilylpropyl)acrylamide (X) and N-propyl-N-(3-triethoxysilylpropyl)acrylamide (Y) by Kobayashi, Journal of Polymer Science, Part A: Polymer Chemistry, 2005, p 2754; 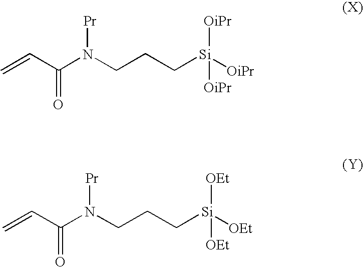
2-hydroxyethyl acrylate (Z) and 2-hydroxyethyl methacrylate (M), by Rozenberg, International Journal of Plastics Technology, 2003, p 17; 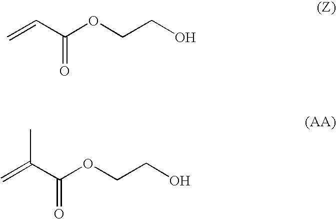
N-butyl acrylate (AB), by Schmitt, Macromolecules, 2001, p 2115
tert-butyl acrylate (AC), by Ishizone, Macromolecules, 1999, p 955;

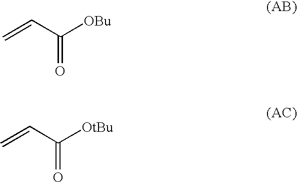

The electron-withdrawing monomer used in the present disclosure can be cyclic or linear. When it is cyclic, the electron-withdrawing group can be, for example, exocyclic, i.e., it does not form an integral part of the cyclic structure of the monomer.

In at least one embodiment of the present disclosure, these monomers exhibit at least two electron-withdrawing groups.

Non-limiting examples of monomers exhibiting at least two electron-withdrawing groups include the monomers of formula (A): 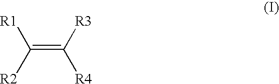
wherein:
R1 and R2 are chosen from, independently of one another, groups with little or no electron-withdrawing effect (with little or no inductive-withdrawing effect), such as:

hydrogen,

saturated or unsaturated, linear, branched or cyclic hydrocarbon groups, for example comprising from 1 to 20 carbon atoms, further for example from 1 to 10 carbon atoms, optionally comprising at least one atom chosen from nitrogen, oxygen and sulphur atom and optionally substituted by at least one group chosen from —OR, —COOR, —COR, —SH, —SR, —OH and halogen atoms,

modified or unmodified polyorganosiloxane residues,

polyoxyalkylene groups,

R3 and R4 are chosen from, independently of one another, electron-withdrawing (or inductive-withdrawing) groups, such as —N(R)₃⁺, —S(R)₂⁺, —SH₂⁺, —NH₃⁺, —NO₂, —SO₂R, —C≡N, —COOH, —COOR, —COSR, —CONH₂, CONHR, —F, —Cl, —Br, —I, —OR, —COR, —SH, —SR and —OH groups, linear or branched alkenyl groups, linear or branched alkynyl groups, C₁-C₄ mono- or polyfluoroalkyl groups, aryl groups, such as phenyl, and aryloxy groups, such as phenoxyloxy,
R is chosen from a saturated or unsaturated and linear, branched or cyclic hydrocarbon group, for example comprising from 1 to 20 carbon atoms, further for example from 1 to 10 carbon atoms, optionally comprising at least one atom chosen from nitrogen, oxygen and sulphur atom and optionally substituted by at least one group chosen from —OR′, —COOR′, —COR′, —SH, —SR′, —OH, halogen atoms, and a residue of a polymer which can be obtained by radical polymerization, by polycondensation or by ring opening, R′ is a C₁-C₁₀ alkyl group.

As used herein, the term “electron-withdrawing group” or “inductive-withdrawing group,” is understood to mean any group which is more electronegative than carbon. For example, reference may be made to the work PR Wells, Prog. Phys. Org. Chem., Vol. 6, 111 (1968).

As used herein, the term “group with little or no electron-withdrawing effect” is understood to mean any group having an electronegativity less than or equal to that of carbon.

The alkenyl or alkynyl groups disclosed above can have, for example, 2 to 20 carbon atoms and even further, for example, from 2 to 10 carbon atoms.

With respect to the saturated or unsaturated, linear, branched or cyclic hydrocarbon groups, mention may be made, for example, of C₁-C₂₀ linear or branched alkyl, alkenyl or alkynyl groups, such as methyl, ethyl, n-butyl, tert-butyl, isobutyl, pentyl, hexyl, octyl, butenyl or butynyl; cycloalkyl groups or aromatic groups.

In addition, non-limiting examples of substituted hydrocarbon groups include hydroxyalkyl and polyhaloalkyl groups.

With respect to the modified and unmodified polyorganosiloxane residues, non-limiting examples of unmodified polyorganosiloxanes include polyalkylsiloxanes, such as polydimethylsiloxanes, polyarylsiloxanes, such as polyphenylsiloxanes, and polyarylalkylsiloxanes, such as polymethylphenylsiloxanes. Non-limiting examples of modified polyorganosiloxanes include polydimethylsiloxanes comprising polyoxyalkylene and/or siloxy and/or silanol and/or amine and/or imine and/or fluoroalkyl groups.

With respect to the polyoxyalkylene groups, non-limiting mention may be made of polyoxyethylene groups and polyoxypropylene groups comprising, for example, 1 to 200 oxyalkylene units.

Non-limiting examples of mono- or polyfluoroalkyl groups disclosed in the definition of R3 and R4 include —(CH₂)ₙ—(CF₂)ₘ—CF₃ and —(CH₂)ₙ—(CF₂)ₘ—CHF₂ where n=1 to 20 and m=1 to 20.

The R1 to R4 substituents can optionally be substituted by a group having a cosmetic activity. The cosmetic activities can be obtained from groups with coloring, antioxidizing, UV-screening and conditioning functions.

Non-limiting examples of groups with a coloring function include azo, quinone, methine, cyanomethine and triarylmethane groups.

Non-limiting examples of groups with an antioxidizing function include butylated hydroxyanisole (BHA), butylated hydroxytoluene (BHT) and vitamin E type groups.

Non-limiting examples of groups with a UV-screening function include benzophenone, cinnamate, benzoate, benzylidenecamphor and dibenzoylmethane type groups.

Non-limiting examples of groups with a conditioning function include cationic and of fatty ester type groups.

In at least one embodiment of the present disclosure, the at least one electrophilic monomer may be chosen from monomers of the family of the cyanoacrylates and their derivatives of formula (II): 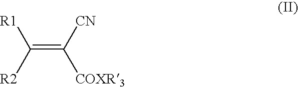
X is chosen from NH, S and O,
R1 and R2 have the same meanings as above, and, for instance, R1 and R2 may both be hydrogen,
R′₃ is chosen from a hydrogen atom or R as defined in the formula (I).

Non-limiting examples of monomers of formula (II) include:
a) monomers belonging to the family of the polyfluoroalkyl 2-cyanoacrylates, such as: the 2,2,3,3-tetrafluoropropyl ester of 2-cyano-2-propenoic acid of formula: 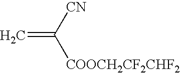
or the 2,2,2-trifluoroethyl ester of 2-cyano-2-propenoic acid of formula: 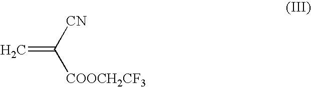
b) alkyl or alkoxyalkyl 2-cyanoacrylates 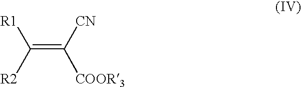
wherein R′₃ is chosen from a C1-C10 alkyl, (C₁-C₄)alkoxy(C₁-C₁₀)alkyl and C₂-C₁₀ alkenyl radical. Non-limiting examples include ethyl 2-cyanoacrylate, methyl 2-cyanoacrylate, n-propyl 2-cyanoacrylate, isopropyl 2-cyanoacrylate, tert-butyl 2-cyanoacrylate, n-butyl 2-cyanoacrylate, isobutyl 2-cyanoacrylate, 3-methoxybutyl cyanoacrylate, n-decyl cyanoacrylate, hexyl 2-cyanoacrylate, 2-ethoxyethyl 2-cyanoacrylate, 2-methoxyethyl 2-cyanoacrylate, 2-octyl 2-cyanoacrylate, 2-propoxyethyl 2-cyanoacrylate, n-octyl 2-cyanoacrylate, isoamyl cyanoacrylate, allyl 2-cyanoacrylate and methoxypropyl 2-cyanoacrylate.

In at least one embodiment, monomers b), i.e., alkyl or alkoxyalkyl 2-cyanoacrylates, are used as the cyanoacrylate monomer(s). In a further embodiment, the cyanoacrylate monomer(s) are chosen from C₆-C₁₀ alkylcyanoacrylates.

In, yet, another embodiment of the present disclosure, the monomers are chosen from the octyl cyanoacrylates of formula (V) and their mixtures: 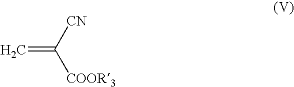
R′₃ is chosen from (CH₂)₇-CH₃, —CH(CH₃)—(CH₂)₅—CH₃,—CH₂—CH(C₂H₅)—(CH₂)₃, —(CH₂)₅—CH(CH₃)—CH₃, and —(CH₂)₄—CH(C₂H₅)—CH₃.

The monomers used in accordance with the present disclosure can be covalently attached to supports, such as polymers, oligomers or dendrimers. The polymer or the oligomer can be linear, branched, of comb structure or of block structure. The distribution of the monomers over the polymer, oligomer or dendrimer structure can be random, in the end position or in the form of blocks.

In at least one embodiment of the present disclosure, the amount of electrophilic monomers, such as cyanoacrylates, ranges from 0.1 to 80% by weight of the total weight of the composition, for example, from 1 to 50%.

According to another embodiment, the electrophilic monomers are monomers capable of polymerizing by the anionic route in the presence of a nucleophilic agent. As used herein, the term “anionic polymerization” is understood to mean the mechanism defined, for example, in the work “Advanced Organic Chemistry”, Third Edition, by Jerry March, pages 151 to 161.

The nucleophilic agents capable of initiating anionic polymerization are systems known per se capable of generating a carbanion on contact with a nucleophilic agent, such as the hydroxyl ions present in water. As used herein, the term “carbanion” is understood to mean the chemical entities, for example, defined in “Advanced Organic Chemistry”, Third Edition, by Jerry March, page 141.

The nucleophilic agents can be applied independently of the composition of the present disclosure. They can also be added to the composition of the present disclosure at the time of use.

The nucleophilic agent can be a molecular compound, an oligomer, a dendrimer or a polymer having nucleophilic functional groups. For example, non-limiting mention of nucleophilic functional groups include: R₂N⁻, NH₂⁻, Ph₃C⁻, R₃C⁻, PhNH⁻, pyridine, ArS⁻, R—C═C⁻, RS⁻, SH⁻, RO⁻, R₂NH, ArO⁻, N₃⁻, OH⁻, ArNH₂, NH₃, I⁻, Br⁻, Cl⁻, RCOO⁻, SCN⁻, ROH, RSH, NCO⁻, CN⁻, NO₃⁻, ClO₄⁻ or H₂O, wherein Ph is a phenyl group, Ar is an aryl group and R is a C₁-C₁₀ alkyl group.

The electrophilic monomers can be synthesized according to known methods described in the art. For example, the cyanoacrylate monomers can be synthesized according to the teaching of U.S. Pat. Nos. 3,527,224, 3,591,767, 3,667,472, 3,995,641, 4,035,334 and 4,650,826.

As used herein, the term “organic solvent” is understood to mean an organic substance capable of dissolving another substance without chemically modifying it.

The at least one liquid organic solvent present in the composition of the present disclosure is chosen from compounds which are liquid at a temperature of 25° C. and at 105 Pa (760 mmHg).

The electrophilic monomers and the organic solvent may be separate compounds.

The organic solvent as used in the dyeing composition of the present disclosure may be chosen, for example, from: aromatic alcohols, such as benzyl alcohol; liquid fatty alcohols, such as C₁₀-C₃₀ fatty alcohols; modified or unmodified polyols, such as glycerol, glycol, propylene glycol, dipropylene glycol, butylene glycol or butyl diglycol; volatile silicones, such as cyclopentasiloxane, cyclohexasiloxane, polydimethylsiloxanes modified or unmodified by alkyl and/or amine and/or imine and/or fluoroalkyl and/or carboxyl and/or betaine and/or quaternary ammonium functional groups; liquid modified polydimethylsiloxanes; mineral, organic or vegetable oils; alkanes, for example C₅ to C₁₀ alkanes; liquid fatty acids; and liquid fatty esters, such as liquid fatty alcohol benzoates or salicylates.

The organic solvent may also be chosen from organic oils; silicones, such as volatile silicones, silicone gums or oils which are or are not aminated and their mixtures; mineral oils; vegetable oils, such as olive oil, castor oil, rapeseed oil, coconut oil, wheat germ oil, sweet almond oil, avocado oil, macadamia oil, apricot oil, safflower oil, candlenut oil, camelina oil, tamanu oil or lemon oil; and organic compounds, such as C₅-C₁₀ alkanes, acetone, methyl ethyl ketone, liquid esters of C₁-C₂₀ acids and of C₁-C₈ alcohols, such as methyl acetate, butyl acetate, ethyl acetate and isopropyl myristate, dimethoxyethane, diethoxyethane, liquid C₁₀-C₃₀ fatty alcohols, such as oleyl alcohol, liquid C₁₀-C₃₀ fatty alcohol esters, such as C₁₀-C₃₀ fatty alcohol benzoates, and their mixtures; polybutene oil, isononyl isononanoate, isostearyl malate, pentaerythrityl tetraisostearate, tridecyl trimelate, the cyclopentasiloxane (14.7% by weight)/polydimethylsiloxane dihydroxylated in the α and ω positions (85.3% by weight) mixture, or their mixtures.

According to one embodiment of the present disclosure, the liquid organic solvent is composed of a silicone or a mixture of silicones, such as liquid polydimethylsiloxanes and liquid modified polydimethylsiloxanes, their viscosity at 25° C. ranging from 0.1 cSt to 1 000 000 cSt, for example from 1 cSt to 30 000 cSt.

Non-limiting examples of the following oils and mixtures of oils used as organic solvents include:

the α,ω-dihydroxylated polydimethylsiloxane/cyclopentadimethylsiloxane (14.7/85.3) mixture sold by Dow Corning under the name of DC 1501 Fluid;

the α,ω-dihydroxylated polydimethylsiloxane/polydimethylsiloxane mixture sold by Dow Corning under the name of DC 1503 Fluid;

the dimethicone/cyclopentadimethylsiloxane mixture sold by Dow Corning under the name of DC 1411 Fluid or that sold by Bayer under the name SF1214;

the cyclopentadimethylsiloxane sold by Dow Corning under the name of DC245 Fluid;

and the respective mixtures of these oils.

In addition to the at least one liquid organic solvent, in at least one embodiment of the present disclosure, the medium of the compositions can comprise water.

For example, the medium of the composition can be anhydrous, that is to say comprising less than 1% by weight of water with respect to the total weight of the composition.

Further, the at least one liquid organic solvent can, in at least one embodiment, be present in an amount ranging from 0.01 to 99% by weight, further for example from 50 to 99% by weight, with respect to the total weight of the composition.

The composition of the present disclosure can be provided in the form of an emulsion and/or be encapsulated, the electrophile monomers being held in an anhydrous medium until the moment of use. When the composition is an emulsion, this emulsion is composed, for example, of a dispersed or continuous phase, which can be composed of water, C₁-C₄ aliphatic alcohols or their mixtures, and an anhydrous organic phase comprising the monomers. In the case of capsules or microcapsules, the capsule can comprise the monomers in an anhydrous medium and be dispersed in an anhydrous medium as defined previously, water, C₁-C₄ aliphatic alcohols or their mixtures.

Polymerization inhibitors, for example, anionic and/or radical polymerization inhibitors, can be introduced into the compositions, in order to increase the stability of the composition over time. Non-limiting mention may be made of the following polymerization inhibitors: sulphur dioxide, nitric oxide, boron trifluoride, hydroquinone and its derivatives, such as hydroquinone monoethyl ether or TBHQ, benzoquinone and its derivatives, such as duroquinone, catechol and its derivatives, such as t-butylcatechol and methoxycatechol, anisole and its derivatives, such as methoxyanisole or hydroxyanisole, pyrogallol and its derivatives, p-methoxyphenol, hydroxybutyltoluene, alkyl sulphates, alkyl sulphites, alkyl sulphones, alkyl sulphoxides, alkyl sulphides, mercaptans, 3-sulphonene and their mixtures. The alkyl groups can be chosen from groups comprising 1 to 6 carbon atoms.

Additionally, inorganic or organic acids may also be used as inhibitors.

Thus, the cosmetic composition according to the present disclosure can also comprise at least one inorganic or organic acid, the latter comprising at least one carboxyl or sulpho group, exhibiting a pKa ranging from 0 to 6, such as phosphoric acid, hydrochloric acid, nitric acid, benzene- or toluenesulphonic acid, sulphuric acid, carbonic acid, hydrofluoric acid, acetic acid, formic acid, propionic acid, benzoic acid, mono-, di- or trichloroacetic acid, salicylic acid, trifluoroacetic acid, octanoic acid, heptanoic acid and hexanoic acid.

The concentration of inhibitor in the cosmetic composition of the present disclosure can range from 10 ppm to 30% by weight and, for example, from 10 ppm to 15% by weight, with respect to the total weight of the composition.

In at least one embodiment, the composition of the present invention can also comprise at least one pigment. It can also comprise metal powders or particles, such as powders or particles formed of aluminium, zinc, copper, and the like.

Furthermore, the composition can also comprise cosmetic active principles commonly used in the art. Non-limiting examples of cosmetic active principles include: fillers, reducing agents, oxidizing agents, fatty substances, silicones, thickening agents, softening agents, antifoaming agents, moisturizing agents, emollients, basifying agents, elastomers, plasticizers, sunscreens, direct nonfluorescent dyes, oxidation dyes, clays, colloidal minerals, fragrances, peptizing agents, preservatives, anionic, cationic, amphoteric, zwitterionic or nonionic surfactants, fixing or nonfixing polymers, conditioning polymers, proteins and vitamins.

These compositions can be provided in various forms, such as lotions, sprays or foams, and can be applied in the shampoo or conditioner form.

In the case of sprays, the composition of the present disclosure can comprise a propellant. The propellant is composed of the compressed or liquefied gases employed for the preparation of aerosol compositions. Non-limiting examples of these gases include: air, carbon dioxide, compressed nitrogen or alternatively a soluble gas, such as dimethyl ether, halogenated (in particular fluorinated) and nonhalogenated hydrocarbons and their mixtures.

According to the process disclosed herein, the composition of the present disclosure is applied to keratinous fibers, such as the hair, in the presence of a nucleophilic agent.

According to at least one embodiment of the process, the nucleophilic agent capable of initiating the polymerization of the cyanoacrylate monomer can be applied beforehand to the keratinous fibers. The nucleophilic agent can be used pure, in solution or in the form of an emulsion or can be encapsulated. It can also be added to the anhydrous composition at the time of use immediately before application to the keratinous fibers.

By way of example, this nucleophilic agent can be water. This water can be contributed, for instance, by preliminary moistening of the keratinous fibers. They can also be added directly to the composition before application.

According to another embodiment of the present disclosure, it is possible to adjust the kinetics of polymerization by moistening the fiber beforehand using an aqueous solution having a pH adjusted using a base, an acid or an acid/base mixture. The acid and/or the base can be inorganic or organic.

According to an alternative form, the process of the present disclosure can be carried out, for example, in several stages: the first stage comprises applying a composition comprising the fluorescent compound or compounds to the fibers, and a second stage comprises applying a composition comprising the electrophilic monomer, the nucleophilic agent being present in the composition comprising the fluorescent compound or in a separate composition, the liquid organic solvent being in either of the compositions.

According to this alternative form, the composition, as disclosed herein, comprising the fluorescent compound or compounds can be, for example, an aqueous solution of fluorescent compounds which makes possible moistening of the fiber and the initiation of the polymerization when the electrophilic monomer is applied.

According to the process of the present disclosure, at least one embodiment comprises either applying the electrophilic monomer and the fluorescent compounds from the same composition or applying, in a first step, the fluorescent compound and then the electrophilic monomer, the organic solvent being present in either of the compositions.

The process of the invention can, also, comprise additional intermediate or final stages, such as the application of a cosmetic product, a rinsing stage or a drying stage. Drying can be carried out with a hood dryer, with a hand-held hairdryer and/or with a smoothing iron. The application of the compositions in accordance with the present disclosure can be followed by a rinsing operation.

It is also possible to carry out multiple applications of the composition of the present disclosure in order to obtain a superimposition of layers in order to achieve specific properties of the deposited material in terms of chemical nature, mechanical strength, thickness, appearance or feel.

In order to improve, inter alia, the adhesion of the poly(cyanoacrylate) formed in situ, the fiber can be pretreated with any type of polymer.

In order to adjust the kinetics of anionic polymerization, it is also possible to increase the nucleophilicity of the fiber by chemical conversion of the keratinous fiber. Non-limiting mention may be made, by way of example, of the reduction of the disulphide bridges of which the keratin is partially composed to give thiols before application of the composition of the present disclosure. Furthermore, non-limiting mention may be made, by way of example, as reducing agents for the disulphide bridges of which the keratin is partially composed, of the following compounds: anhydrous sodium thiosulphate, powdered sodium metabisulphite, thiourea, ammonium sulphite, thioglycolic acid, thiolactic acid, ammonium thiolactate, glyceryl monothioglycolate, ammonium thioglycolate, thioglycerol, 2,5-dihydroxybenzoic acid, diammonium dithioglycolate, strontium thioglycolate, calcium thioglycolate, zinc formaldehyde sulphoxylate, isooctyl thioglycolate, d,l-cysteine and monoethanolamine thioglycolate.

The application of the composition according to the present disclosure can also be preceded by a hair treatment, such as direct or oxidation dyeing.

According to the present disclosure, the monomers may be chosen from monomers capable of polymerizing on keratinous fibers under cosmetically acceptable conditions. For instance, the polymerization of the monomer can be carried out at a temperature of less than or equal to 80° C., which does not prevent the application being terminated by drying with a hood dryer, blow drying, subjecting to a flat iron or subjecting to a curling tong.

According to one embodiment of the process, as disclosed herein, the composition of the present disclosure is applied to dark hair. Therefore, the cosmetic composition of the disclosure makes it possible to dye, with a lightening effect, artificially pigmented or colored hair having a tone height of less than or equal to 6, for instance less than or equal to 4. The tone height is a well known characteristic of the art of dyeing. It is, for example, described in the work “Science des traitements capillaires” [Hair Treatment Science], Charles Zviak, published by Masson, 1988, page 278.

Another aspect of the present disclosure is a dyeing kit comprising a first compartment which comprises at least one soluble fluorescent compound and a second compartment which comprises at least one electrophilic monomer and, optionally, a third compartment which comprises the nucleophilic agent, the organic solvent being present in one and/or other of the three compartments. According to another embodiment of the dyeing kit, the at least one soluble fluorescent compound and the at least one electrophilic monomer are present in the same composition in at least one first compartment, a nucleophilic agent is present in an optional second compartment, and the organic solvent is present in one and/or other of the compartments. In a further embodiment, the composition comprising both the at least one soluble fluorescent compound and the at least one electrophilic monomer is anhydrous.

According to a first alternative form, the kit comprises a first composition which comprises the at least one soluble fluorescent compound and a second composition which comprises the at least one electrophilic monomer and the at least one liquid organic solvent. According to one embodiment of this alternative form, the composition comprising the least one fluorescent compound is an aqueous composition and the composition comprising the at least one electrophilic monomer is an anhydrous composition.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, the numerical values set forth in the specific examples are reported as precisely as possible. Any numerical value, however, inherently contain certain errors necessarily resulting from the standard deviation found in their respective testing measurements.

The following examples are intended to illustrate the invention in a non-limiting manner.

### EXAMPLES

### Example 1

### Family of the Pyrazines

The following composition was prepared:

| Ingredient | Amount |
|---|---|
| α,ω-Dihydroxylated polydimethylsiloxane/ | 40 g |
| cyclopentadimethylsiloxane (14.7/85.3), sold by Dow Corning | |
| under the name of DC 1501 Fluid | |
| Cyclopentadimethylsiloxane, sold by Dow Corning | 40 g |
| under the name of DC245 Fluid | |
| Compound 1: 5-chloro-8-benzylminopyrazino[2,3-d]pyridazine* | 10 g |
| Methylheptyl cyanoacrylate, manufactured by Chemence | 10 g |
| Acetic acid | 0.25 g |

*cited in the following publications: Natu R. Patel, Journal of Heterocyclic Chemistry, Vol. 3, No. 4, 1966, pp 512-517; Kazuko Shirai, Journal of the Society of Dyes and Colorists, Vol. 114, No. 12, December 1998, pp 368-374.

0.5 g of this composition was applied to a lock of clean and wet natural chestnut hair weighing 1 g and with a tone height of 4. After a leave-in time of 15 minutes at ambient temperature, the lock was dried with a hair dryer for 2 minutes.

The lock was colored intense orange-yellow and the coloring obtained was resistant to shampooing operations. The coloring was highly fluorescent to daylight. The lock obtained exhibited a soft feel.

### Example 2

### Family of the Pyrazines

The following composition was prepared:

| Ingredient | Amount |
|---|---|
| α,ω-Dihydroxylated polydimethylsiloxane/ | 40 g |
| cyclopentadimethylsiloxane (14.7/85.3), sold by Dow Corning | |
| under the name of DC 1501 Fluid | |
| Cyclopentadimethylsiloxane, sold by Dow Corning under the | 40 g |
| name of DC245 Fluid | |
| Compound 2: 2,3-dicyano-5-hydroxy-6- | 10 g |
| (2-julolidinylethenyl)pyrazine* | |
| Methylheptyl cyanoacrylate, manufactured by Chemence | 10 g |

*cited in the following publications: Natu R. Patel, Journal of Heterocyclic Chemistry, Vol. 3, No. 4, 1966, pp 512-517; Kazuko Shirai, Journal of the Society of Dyes and Colorists, Vol. 114, No. 12, December 1998, pp 368-374.

0.5 g of this composition was applied to a lock of clean and wet natural chestnut hair weighing 1 g and with a tone height of 4. After a leave-in time of 15 minutes at ambient temperature, the lock was dried with a hair dryer for 2 minutes.

The lock was colored yellow and the coloring obtained was resistant to shampooing operations. The coloring was highly fluorescent when irradiated with daylight. The hair had a lightened appearance compared with the starting chestnut hair. The dyeing composition exhibited good stability over time.

### Example 3

### Family of the Coumarins

The following composition was prepared:

| Ingredient | Amount |
|---|---|
| α,ω-Dihydroxylated polydimethylsiloxane/ | 40 g |
| cyclopentadimethylsiloxane (14.7/85.3), sold by Dow Corning | |
| under the name of DC 1501 Fluid | |
| Cyclopentadimethylsiloxane, sold by Dow Corning under | 40 g |
| the name of DC245 Fluid | |
| Compound 5: diethylaminocoumarin from CIBA | 10 g |
| Methylheptyl cyanoacrylate, from Chemence | 10 g |
| Acetic acid | 0.25 g |

0.5 g of this composition was applied to a lock of clean and wet natural chestnut hair weighing 1 g and with a tone height of 4. After a leave-in time of 15 minutes at ambient temperature, the lock was dried with a hair dryer for 2 minutes.

The lock was colored red, slightly bluish, and the coloring obtained was resistant to shampooing operations. The coloring was highly fluorescent to daylight. The visual appearance and the feel of the lock obtained were highly satisfactory.

Evaluation

The color of the locks was evaluated before and after application of the composition of the disclosure in the L*a*b* system using a Minolta® DM3600d spectrophotometer. In this L*a*b* system, L* represents the intensity of the color, a* indicates the green/red color axis and b* indicates the blue/yellow color axis. The lower the value of L, the darker the color or the more intense it is. The higher the value of a*, the redder the hue and, the higher the value of b*, the bluer the hue.

The results are listed in the following table:

| Examples | L* | a* | b* |
|---|---|---|---|
| Lock with a tone height of 4 | 22.8 | 2.9 | 3.6 |
| Ex. 1: Compound No. 1 | 39.3 | −8.2 | 14.9 |
| pyrazine | | | |
| Ex. 2: Compound No. 2 | 31.5 | 5.4 | 25.5 |
| pyrazine | | | |
| Ex. 3: Diethylaminocoumarin | 23.4 | 5.2 | −5.1 |

These results show that, after application of compositions 1 and 2 of the present disclosure, the value of L*, representative of the intensity of the color of the locks, is greater than that obtained on the chestnut hair with a tone height of 4. This shows the lightening effect of the composition of the present disclosure without it being necessary to have recourse to bleaching of the keratinous fibers. Furthermore, persistent colorings are thus obtained. The lock of hair colored with diethylaminocoumarin exhibits a highly visible red color.

### Example 4

### Application in Two Stages

The following composition A was prepared:

| Ingredient | Amount |
|---|---|
| 2-[2-(4-(Dimethylamino)phenyl)ethenyl]-1-ethylpyridinium | 5 g |
| iodide, sold under the name Photosensitizing Dye NK-557 | |
| by Ubichem | |
| Water | 95 g |

The following composition B was prepared:

| Ingredient | Amount |
|---|---|
| α,ω-Dihydroxylated polydimethylsiloxane/ | 45 g |
| cyclopentadimethylsiloxane (14.7/85.3), sold by Dow Corning | |
| under the name of DC 1501 Fluid | |
| Cyclopentadimethylsiloxane, sold by Dow Corning under the | 44.75 g |
| name of DC245 Fluid | |
| Methylheptyl cyanoacrylate, from Chemence | 10 g |
| Acetic acid | 0.25 g |

0.5 g of the composition A was applied to a lock of clean and wet natural white hair weighing 1 g. 0.5 g of the composition B was then applied to the lock. After leaving at ambient temperature for 15 minutes, the lock was dried with a hair dryer for 2 minutes.

The lock was colored yellow and the coloring obtained was resistant to shampooing operations. The coloring was fluorescent when irradiated with daylight. The visual appearance and the feel of the lock obtained were highly satisfactory. Persistent coloring were thus obtained.

What is claimed is:

1. A dyeing composition comprising, in an medium suitable for dyeing, at least one electrophilic monomer, at least one soluble fluorescent compound and at least one liquid organic solvent.

2. A composition according to claim 1, wherein the at least one electrophilic monomer is chosen from monomers of formula (I): 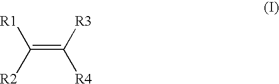
wherein:
R1 and R2 are chosen from, independently of one another, groups with little or no electron-withdrawing effect, and
R3 and R4 are chosen from, independently of one another,electron-withdrawing groups.

3. A composition according to claim 2, wherein in formula (I) R1 and R2 independently are chosen from
hydrogen;
saturated or unsaturated, linear, branched or cyclic hydrocarbon groups optionally comprising at least one atom chosen from nitrogen, oxygen and sulphur atoms, and optionally substituted by at least one group chosen from —OR, —COOR, —COR, —SH, —SR, —OH and halogen atoms;
modified or unmodified polyorganosiloxane residues; and
polyoxyalkylene groups,
wherein R is chosen from a saturated or unsaturated, linear, branched or cyclic hydrocarbon group comprising from 1 to 20 carbon atoms and optionally comprising at least one atom chosen from nitrogen, oxygen and sulphur atoms and optionally substituted by at least one group chosen from —OR′, —COOR′, —COR′, —SH, —SR′, —OH, halogen atoms, and a residue of a polymer, and R′ is a C₁-C₁₀ alkyl group.

4. A composition according to claim 3, wherein R1 and R2 independently are chosen from saturated or unsaturated, linear, branched or cyclic hydrocarbon groups comprising from 1 to 20 carbon atoms.

5. A composition according to claim 2, wherein, in formula (I), R3 and R4 are chosen independently from —N(R)₃⁺, —S(R)₂⁺, —SH₂⁺, —NH₃⁺, —NO₂, —SO₂R, —C≡N, —COOH, —COOR, —COSR, —CONHR, —CONH₂, —F, —Cl, —Br, —I, —OR, —COR, —SH, —SR and —OH groups, linear or branched alkenyl groups, linear or branched alkynyl groups, C₁-C₄ mono- or polyfluoroalkyl groups, aryl groups and aryloxy groups,
wherein R is chosen from saturated or unsaturated, linear, branched or cyclic hydrocarbon groups comprising from 1 to 20 carbon atoms, optionally comprising at least one atom chosen from nitrogen, oxygen and sulphur atoms and optionally substituted by at least one group chosen from —OR′, —COOR′, —COR′, —SH, —SR′, —OH, halogen atoms, and a residue of a polymer, R′ being a C₁-C₁₀ alkyl radical.

6. A composition according to claim 1, wherein the at least one electrophilic monomer is chosen from cyanoacrylate monomers of formula (II): 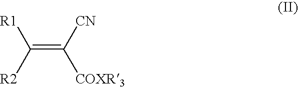
wherein
X is chosen from NH, S and O,
R′₃ is chosen from a hydrogen atom and R,
R1 and R2 independently are chosen from
hydrogen;
saturated or unsaturated, linear, branched or cyclic hydrocarbon groups optionally comprising at least one atom chosen from nitrogen, oxygen and sulphur atoms, and optionally substituted by at least one group chosen from —OR, —COOR, —COR, —SH, —SR, —OH and halogen atoms;
modified or unmodified polyorganosiloxane residues; and
polyoxyalkylene groups,
wherein R is chosen from a saturated or unsaturated, linear, branched or cyclic hydrocarbon group comprising from 1 to 20 carbon atoms and optionally comprising at least one atom chosen from nitrogen, oxygen and sulphur atoms and optionally substituted by at least one group chosen from —OR′, —COOR′, —COR′, —SH, —SR′, —OH, halogen atoms, and a residue of a polymer, and R′ is a C₁-C₁₀ alkyl group.

7. A composition according to claim 3, wherein R1 and R2 are both hydrogen.

8. A composition according to claim 6, wherein the cyanoacrylate monomers are chosen from monomers of formula (IV): 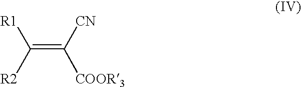
wherein
R′₃ is chosen from C₁-C₁₀ alkyl, (C₁-C₄)alkoxy(C₁-C₁₀)alkyl, and C₂-C₁₀ alkenyl radicals and
R1 and R2 independently are chosen from
hydrogen;
saturated or unsaturated, linear, branched or cyclic hydrocarbon groups optionally comprising at least one atom chosen from nitrogen, oxygen and sulphur atoms, and optionally substituted by at least one group chosen from —OR, —COOR, —COR, —SH, —SR, —OH and halogen atoms;
modified or unmodified polyorganosiloxane residues; and
polyoxyalkylene groups,
wherein R is chosen from a saturated or unsaturated, linear, branched or cyclic hydrocarbon group comprising from 1 to 20 carbon atoms and optionally comprising at least one atom chosen from nitrogen, oxygen and sulphur atoms and optionally substituted by at least one group chosen from —OR′, —COOR′, —COR′, —SH, —SR′, —OH, halogen atoms, and a residue of a polymer, and R′ is a C₁-C₁₀ alkyl group.

9. A composition according to claim 8, wherein R′₃ is an alkyl radical comprising from 6 to 10 carbon atoms.

10. A composition according to claim 8, wherein R1 and R2 are both hydrogen.

11. A composition according to claim 1, wherein the at least one electrophilic monomer is an alkyl cyanoacrylate of formula: 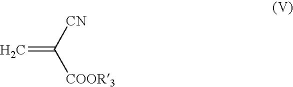
wherein R′₃ is chosen from —(CH₂)₇—CH₃, —CH(CH₃)—(CH₂)₅—CH₃, —CH₂—CH(C₂H₅)—(CH₂)₃—CH₃, —(CH₂)₅—CH(CH₃)—CH₃, and —(CH₂)₄—CH(C₂H₅)—CH₃.

12. A composition according to claim 1, wherein the at least one electrophilic monomer is present in an amount ranging from 0.1 to 80% by weight relative to the total weight of the composition.

13. A composition according to claim 1, wherein the at least one soluble fluorescent compound is chosen from fluorescent dyes and optical brighteners.

14. A composition according to claim 13, wherein the fluorescent dyes exhibit a maximum emission wavelength in orange-coloured visible light.

15. A composition according to claim 13, wherein the optical brightener or brighteners exhibit a maximum emission wavelength in blue visible light.

16. A composition according to claim 13, wherein the fluorescent dyes are chosen from dyes with the following structures:

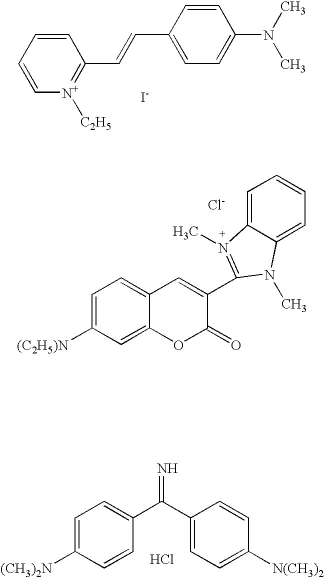

17. A composition according to claim 13, wherein the at least one fluorescent compound is chosen from naphthalimides, coumarins, xanthenes, rhodamines, thioxanthenes, naphtholactams, azalactones, methines, oxazines and thiazines, distyrylbenzenes, distyrylbiphenyls, triazinylaminostilbenes, stilbazoliums and their dimers, their trimers and their tetramers, bis(benzoxazoles), cationic or noncationic bis(benzimidazoles), anionic or nonanionic diphenylpyrazolines, diketopyrrolopyrroles, dicyanopyrazines and the derivatives of all these families.

18. A composition according to claim 1, wherein the at least one fluorescent compound is present (for each) in an amount ranging from 0.01 to 40% by weight relative to the total weight of the composition.

19. A composition according to claim 1, wherein the composition is anhydrous.

20. A composition according to claim 1, wherein the at least one liquid organic solvent is chosen from organic oils, silicones, mineral oils, vegetable oils, C₅-C₁₀ alkanes, acetone, methyl ethyl ketone, liquid esters of C₁-C₂₀ acids and of C₁-C8 alcohols, dimethoxyethane, diethoxyethane, liquid C₁₀-C₃₀ fatty alcohols, liquid C₁₀-C₃₀ fatty alcohol esters, polybutene oil, isononyl isononanoate, isostearyl malate, pentaerythrityl tetraisostearate, tridecyl trimelate, the cyclopentasiloxane (14.7% by weight)/polydimethylsiloxane dihydroxylated in the a and w positions (85.3% by weight) mixture, and mixtures thereof.

21. A composition according to claim 1, further comprising at least one nucleophilic agent.

22. A composition according to claim 21, wherein the at least one nucleophilic agent is water.

23. A process for dyeing keratinous fibers comprising applying to said fibers, in the presence of a nucleophilic agent, a dyeing composition comprising, in a medium suitable for dyeing, at least one electrophilic monomer, at least one soluble fluorescent compound and at least one liquid organic solvent

24. A process according to claim 23, wherein the nucleophilic agent is water.

25. A process for dyeing keratinous fibers according to claim 23, comprising a first stage comprising applying a composition comprising at least one fluorescent compound to the fibers, and a second stage comprising applying a composition comprising at least one electrophilic monomer to the fibers, the nucleophilic agent being present in the composition comprising the at least one fluorescent compound or in a separate composition.

26. A process according to claim 25, wherein the composition comprising the at least one fluorescent compound is an aqueous composition and the composition which comprises the at least one electrophilic monomer is anhydrous.

27. A process according to claim 23 for the dyeing of keratinous fibers exhibiting a tone height of less than 6.

28. A process according to claim 27, further comprising a tone height of less than or equal to 4.

29. A dyeing kit comprising a first compartment which comprises at least one soluble fluorescent compound, a second compartment comprising at least one electrophilic monomer and optionally a third compartment which comprises a nucleophilic agent, wherein at least one liquid organic solvent is present in at least one of the three compartments.

30. A dyeing kit comprising a first composition comprising at least one fluorescent compound and at least one electrophilic monomer, and optionally a second composition which comprises a nucleophilic agent, wherein at least one liquid organic solvent is present in at least one of the compositions.

31. A dyeing kit according to claim 30, wherein said composition comprising both the at least one soluble fluorescent compound and the at least one electrophilic monomer is anhydrous.
